(19) United States
(12) Patent Application Publication
(10) Pub. No.: US 2015/0087075 A1
(43) Pub. Date: Mar. 26, 2015

(54) SULPHUR DETECTION METHOD AND KIT

(71) Applicant: SHELL OIL COMPANY, Houston, TX (US)

(72) Inventors: Majid Jamshed Chughtai, Ince Chester (GB); Jacques Colange, Colombes Cedex (FR); Hadyn Howard Jones, Ince Chester (GB); Fabien Phillippe Didier Surmont, Nanterre Cedex (FR)

(21) Appl. No.: 14/389,401

(22) PCT Filed: Mar. 14, 2013
(86) PCT No.: PCT/EP2013/055208

§ 371 Date: Sep. 30, 2014

(30) Foreign Application Priority Data
Apr. 2, 2012 (EP) 12305394.4

(51) Int. Cl. G01N 33/38; G01N 33/42; G01N 21/82; G01N 31/02

(52) U.S. Cl. CPC G01N 33/383; G01N 31/02; G01N 33/42; G01N 21/82
USPC 436/123; 422/82.09

(57) ABSTRACT

Method of detecting the presence of sulphur in a construction material such as asphalt or concrete comprising the steps of:·(a) adding a quantity of the construction material to a solvent to form a test solution, wherein the solvent is capable of solubilising all or part of any sulphur which is present in the construction material and wherein the solvent is miscible with an aqueous solution; and·(b) detecting the presence of sulphur in the test solution by (i) adding to the solution a reagent which induces part or all of any sulphur present in the test solution to precipitate and (ii) measuring the turbidity of the test solution after the precipitation has been formed. The present invention further relates to a sulphur detection kit for detecting the presence of sulphur in construction materials such as asphalt or concrete.

### FIELD OF THE INVENTION

The invention relates to a method for detecting the presence of sulphur in construction materials such as asphalt and concrete. The invention also relates to a sulphur detection kit for detecting the presence of sulphur in construction materials such as asphalt and concrete.

### BACKGROUND OF THE INVENTION

In the road construction and road paving industry, it is a well-practised procedure to coat aggregate material such as sand, gravel, crushed stone or mixtures thereof with hot fluid bitumen, spread the coated material as a uniform layer on a road bed or previously built road while it is still hot, and compact the uniform layer by rolling with heavy rollers to form a smooth surfaced road.

The combination of bitumen with aggregate material, such as sand, gravel, crushed stone or mixtures thereof, is referred to as “asphalt”. Bitumen, also referred to as “asphalt binder”, is usually a liquid binder comprising asphaltenes, resins and solvents. Bitumen can for example comprise pyrogenous mixtures derived from petroleum residues such as residual oils, tar or pitch or mixtures thereof.

It is known in the art that sulphur can be mixed with bitumen for applications in the road construction and road paving industry. Sulphur-modified bitumen is formulated by replacing some of the bitumen in conventional binders by elemental sulphur. Sulphur can be incorporated into an asphalt mixture in the form of solid pellets, for example, such as those commercially available from Shell under the tradename Thiopave®.

Thiopave® is a sulphur based binder which can be used as a partial bitumen substitute in asphalt mixtures to produce sulphur asphalt for paving applications. Thiopave® pellets can replace up to 25% of the bitumen content, by volume, in a conventional asphalt mixture. It is incorporated into an asphalt mixing sequence after the aggregate and bitumen have been added and it is the residual heat energy of this mixture which causes the pellets to melt. It is of the utmost importance when using Thiopave® to ensure that production temperatures do not exceed 145° C. otherwise there is considerable risk of hydrogen sulphide (H₂S) formation and release.

Driven by customer demand for sustainable products, asphalt companies are evaluating means to reduce their reliance on virgin aggregate supplies. Increasingly, the use of recycled asphalt planings (RAPs) as part of the asphalt formulation is viewed as part of the solution. RAP is a means to recycle aggregate by crushing or milling asphalt which may be in the form of planings (extracted from an asphalt pavement), off-specification asphalt or returned loads. RAP can be used as part of an asphalt formulation or as a substitute for virgin aggregate. Regional asphalt specifications define the allowable RAP content in asphalt mixtures. As the aggregate in RAP is already coated in binder, its usage implies a lower fresh binder content in an asphalt formulation compared to an asphalt mixture comprising wholly virgin aggregate. RAP is incorporated into an asphalt mixture along with the aggregate prior to the addition of the bitumen/binder.

Future applications will require the use of sulphur asphalt as RAP and therefore, it is important that consideration is given to the sustainability aspects of sulphur-containing asphalt, in particular, whether it can be readily recycled and its use as RAP.

As sulphur asphalt needs to be kept below 145° C., asphalt mixtures containing sulphur asphalt based RAP need to be processed at lower temperatures than asphalt mixtures containing conventional asphalt based RAP. Therefore, the ability to distinguish between conventional asphalt based RAP and a sulphur asphalt based RAP is of the greatest importance.

Currently, there is nothing commercially available which can be used to detect whether a pavement comprises regular asphalt or sulphur-modified asphalt. In the future, it may be possible to keep track of where these sulphur asphalt pavements are being placed. If it were known precisely where sulphur-asphalt was laid, then it would be known exactly which recycling process to use. However, this would place a heavy burden on the keeping of precise records as to where such sulphur-modified asphalts have been placed.

The present inventors have developed a method of detecting sulphur in sulphur-containing materials, such as sulphur-containing construction materials, for example asphalt or concrete. The present inventors have also developed a sulphur detection kit for detecting whether sulphur is present in sulphur-containing materials such as asphalt or concrete.

### SUMMARY OF THE INVENTION

According to the present invention there is provided a method of detecting the presence of sulphur in a construction material such as asphalt or concrete comprising the steps of:

(a) adding a quantity of the construction material to a solvent to form a test solution, wherein the solvent is capable of solubilising all or part of any sulphur which is present in the construction material and wherein the solvent is miscible with an aqueous solution; and

(b) detecting the presence of sulphur in the test solution by

(i) adding to the solution a reagent which induces part or all of any sulphur present in the test solution to precipitate; and

(ii) measuring the turbidity of the test solution after the precipitate has been formed.

According to another aspect of the present invention there is provided a sulphur detection kit for detecting the presence of sulphur in construction materials such as asphalt or concrete wherein the sulphur detection kit comprises:

(a) solvent wherein the solvent is capable of solubilising part or all of any sulphur which is present in the construction material to form a test solution and wherein the solvent is miscible with an aqueous solution; and

(b) means for detecting the presence of sulphur in the test solution, comprising:

(i) a reagent which induces part or all of any sulphur present in the test solution to precipitate to form a turbid solution.

### DETAILED DESCRIPTION OF THE INVENTION

The solvent for use in the present invention can be any solvent which is capable of solubilising part or all of any sulphur which is present in the material to be tested. The solvent for use in the present invention must also be miscible with aqueous solutions.

A suitable solvent for use herein is an alcoholic solvent, preferably selected from isopropanol, ethanol, propanol, n-butanol, sec-butanol, t-butanol, and mixtures thereof. In a particularly preferred embodiment of the present invention, the solvent is isopropanol.

The solvent is used for solubilising all or part of any sulphur which is present in the material to be tested to form a test solution.

A typical quantity of asphalt or concrete for use in the method and kit herein lies in the range of from 1 g to 100 g, preferably from 2 g to 80 g, more preferably from 3 g to 50 g, and especially from 5 g to 20 g.

The volume of the alcoholic solvent for use in the present invention is preferably in the range of from 1 mL to 500 mL, more preferably in the range of from 3 mL to 200 mL, even more preferably in the range of from 5 mL to 40 mL.

In the sulphur detection method of the present invention, there comprises step (b):

(b) detecting the presence of sulphur in the test solution by

(i) adding to the test solution a reagent which causes part or all of any sulphur present in the test solution to precipitate; and

(ii) measuring the turbidity of the test solution after the precipitate has been formed.

The sulphur detection kit comprises a means for detecting the presence of sulphur in the test solution comprising

(i) a reagent which induces part or all of the sulphur present in the test solution to precipitate.

Any reagent which can induce part or all of the sulphur present in the test solution to precipitate out of solution is suitable for use herein. Such reagent should be miscible with the solvent which is capable of solubilising all or part of the sulphur which is present in the construction material. The reagent is preferably water or an aqueous salt solution. Suitable salts for use in the aqueous salt solution include, but are not necessarily limited to alkali metal halides, alkaline earth metal halides, alkali metal sulphates, alkaline earth metal sulphates, alkali metal nitrates, alkaline earth metal nitrates, alkali metal carbonates, alkaline earth metal carbonates, and the like, and mixtures thereof, more preferably alkali metal halides and alkaline earth metal halides.

Suitable alkaline earth metal halides for use in the aqueous solution herein are selected from calcium chloride, magnesium chloride, and mixtures thereof.

Suitable alkali metal halides for use in the aqueous solution herein are selected from lithium chloride, sodium chloride, potassium chloride, and mixtures thereof.

In preferred embodiments of the present invention, the aqueous salt solution is an alkaline earth metal halide, preferably calcium chloride.

It is preferred that the aqueous salt solution has a concentration in the range of from 0.0001M to 1.0M, more preferably from 0.0005M to 0.1M and even more preferably from 0.0007M to 0.07M.

When all or part of the sulphur present in the test solution has precipitated out of solution, the test solution becomes cloudy or turbid. The cloudy appearance of a solution is caused by the presence of a suspension of a precipitate. Solutions free of these contaminants are clear to the naked eye and have low turbidity. Conversely, cloudy solutions have a high turbidity.

While not wishing to be limited by theory, it is believed that the present invention takes advantage of the solvation characteristics of elemental sulphur. As sulphur is sparingly soluble in an alcohol such as isopropanol, treating an alcoholic extract of an asphalt sample with an aqueous salt solution such as calcium chloride causes the sulphur to become insoluble.

Measuring the turbidity of the test solution after the precipitate has been formed may be achieved by visual inspection. Alternatively, the measurement may be achieved using a device such as a nephelometer or a turbidity meter.

An optional element of the test kit herein is:

(ii) means for measuring the turbidity of the solution after the sulphur has precipitated.

In one embodiment of the present invention, the sulphur detection kit does not contain a means for measuring the turbidity of the test solution after the sulphur has precipitated, but instead includes instructions to the user instructing them to use such a means for measuring the turbidity of the test solution after the sulphur has precipitated.

A preferred means for measuring the turbidity of the test solution after the sulphur has precipitated is a nephelometer or a turbidity meter. In preferred embodiments of the present invention, the nephelometer or turbidity meter used herein is portable.

Turbidity can be measured in several ways. In the present invention, the measurement of turbidity is preferably based on light scattering. In the light scattering method a light beam is passed through a sample cell; some of this light passes through unhindered however some light waves collide with particulates causing it to deviate from its path. Photodetectors placed surrounding the sample cell collect the light beams and the relative intensity of the light passing directly through the sample cell and the scattered light gives a measure of the turbidity. The unit of measure is the Formazin Nephelometric Unit (FNU) (in the ISO 7027 method) or the Formazin Turbidity Unit (FTU). Suitable turbidity meters are commercially available, e.g. Hach 2100Qis Portable Turbidimeter.

In the method of the present invention, the turbidity of the test solution is measured after a certain time period has elapsed following the addition of the test solution to the reagent which causes part or all of any sulphur present in the test solution to precipitate. Preferably the turbidity of the test solution is measured between 2 and 20 minutes, more preferably between 2 and 15 minutes, even more preferably between 2 and 10 minutes, and especially between 5 and 10 minutes, after the addition of the test solution to the reagent which causes part or all of any sulphur present in the test solution to precipitate.

In the present invention, the presence of sulphur in asphalt and concrete is indicated by the test solution preferably having a turbidity of 150 FNU or greater after all or part of the sulphur has precipitated out of solution.

In a particularly preferred embodiment of the present invention, the sulphur detection kit is portable such that it may be easily transported to the site to be tested, e.g. asphalt pavement or concrete structure.

In a preferred embodiment of the present invention, the sulphur detection kit comprises the following elements:

(1) Container, containing the solvent which is (i) capable of solubilising part or all of any sulphur which is present in the construction material to be tested to form a test solution and which is (ii) miscible with aqueous solutions; and
(2) Turbidity cell containing the reagent which causes part or all of the sulphur present in the test solution to precipitate; and optionally
(3) pipette (e.g. 3m1 plastic disposable pipette) for transferring a portion of the test solution to the turbidity cell.

In a preferred embodiment of the present invention the sulphur detection kit comprises instructions for using the test kit to carry out the sulphur detection method of the present invention.

The sulphur detection method and kit of the present invention can be used to detect the presence of sulphur in an asphalt pavement or concrete structure and hence it can be determined how that pavement or concrete may be recycled.

The invention will now be illustrated by means of the following Examples, which are not intended to limit the scope of the invention.

### EXAMPLES

### Examples 1-25

A wide range of asphalt samples were assessed for the presence of sulphur using the method and test kit of the present invention. Compositional details of the asphalt samples tested in terms of wt % sulphur, wt % aggregate and wt % bitumen are shown in Table 1 below.

A sulphur detection kit containing the following equipment was used to carry out the sulphur detection method of the present invention:

1. Plastic jar, containing 30 ml IPA.
2. Turbidity cell, containing 10 ml 0.001M CaCl₂ (aq).
3. 3 ml plastic disposable pipette.

The procedure required weighing a 10 g+1 g sample (approximately 3 to 4 small pieces) of each asphalt to be tested into a plastic jar containing 30 ml isopropanol (IPA). The sample and the IPA was continuously mixed for 2 minutes by swirling. After 2 minutes 6m1 of the resulting solution was transferred into a turbidity cell containing 10 ml 0.001M CaCl₂ (aq). The cell was sealed and shaken vigorously for 10 seconds. The cell was placed in a turbidity meter (Hach 2100Qis Portable Turbidimeter). After 5 minutes the read button was pressed and the turbidity result was noted. If the reading was close to 200 FNU (±40 FNU) then a further reading was taken after another 5 minutes. If after 5 minutes the turbidity reading was significantly higher or lower than 200 FNU then the test was stopped. If the turbidity reading was within ±40 FNU then a further reading was taken after another 5 minutes. The turbidity readings are shown in Table 1 below.

From the data in Table 1 it can be seen that all sulphur containing samples had turbidity values greater than 200 FNU (Formazin Nephelometric Unit) whilst the sulphur free samples displayed values below 130 FNU.

TABLE 1

| | | | | | Final |
|---|---|---|---|---|---|
| | Sample | Sulphur | Bitumen | Aggregate | Turbidity |
| | Mass | Content | content | content | Reading |
| Example | (g) | wt % | wt % | wt % | (FNU) |
| 1 | 10.9 | 3.1 | 3.1 | 93.8 | 605 |
| 2 | 10.4 | 0 | 4.8 | 95.2 | 84 |
| 3 | 10.6 | 0 | 7.7 | 92.3 | 129 |
| 4 | 10.0 | 4 | 2.0 | 94.3 | 320 |
| 5 | 10.1 | 3.8 | 1.9 | 94.6 | 228 |
| 6 | 10.0 | 3.6 | 1.8 | 95.0 | 331 |
| 7 | 10.9 | 0.5 | 4.5 | 94.7 | 255 |
| 8 | 10.3 | 0.5 | 4.5 | 94.7 | 228 |
| 9 | 10.0 | 1.1 | 4.2 | 94.4 | 237 |
| 10 | 10.4 | 1.7 | 3.9 | 94.1 | 248 |
| 11 | 10.0 | 2.4 | 3.5 | 95.2 | 330 |
| 12 | 10.2 | 0 | 4.8 | 94.4 | 99 |
| 13 | 11.0 | 0 | 4.8 | 94.4 | 27 |
| 14 | 10.6 | 1.7 | 3.9 | 94.4 | 781 |
| 15 | 10.2 | 1.7 | 3.9 | 94.4 | 599 |
| 16 | 9.7 | 1.7 | 3.9 | 94.4 | 803 |
| 17 | 10.7 | 1.7 | 3.9 | 94.4 | 212 |
| 18 | 10.8 | 1.7 | 3.9 | 94.4 | 899 |
| 19 | 10.4 | 1.4 | 3.9 | 94.4 | 240 |
| 20 | 10.4 | 1.4 | 3.2 | 95.4 | 352 |
| 21 | 10.9 | 1.4 | 3.2 | 95.4 | 220 |
| 22 | 10.3 | 2.3 | 3.4 | 94.3 | 443 |
| 23 | 10.2 | 0 | 5.0 | 95.0 | 32 |
| 24 | 10.6 | 0.4 | 4.4 | 95.2 | 252 |
| 25 | 12.3 | 0 | 4.7 | 95.3 | 37 |

To investigate the nature of the precipitate formed, a sample of the precipitate was analysed using X-ray diffraction and scanning electron microscopy. The precipitate from Example 18 was collected by pipette into petri dishes and the liquid allowed to evaporate in air. This left a small amount of white deposit which was dispersed onto a low background substrate for X-ray diffraction (XRD) analysis, with a further amount of sample secured onto aluminium stubs for analysis in the scanning electron microscope (SEM).

### Scanning Electron Microscopy Results

Table 2 below shows the SEM/Energy dispersive x-ray spectroscopy (SEM/EDX) analysis results of the deposit from Example 18.

TABLE 2

| Abundance Levels | Element |
|---|---|
| Relatively High wt % | Carbon |
| Moderate to High wt % | Sulphur |
| Low to Moderate wt % | Oxygen |
| Low wt % | Silicon |
| Low to Trace wt % | Calcium |
| Trace wt % | Sodium, Magnesium, |
| | Aluminium, Chlorine, |
| | Potassium, Iron |

Table 2 shows that the deposit from Example 18 contained a high amount of carbon and sulphur. The carbon (and to a lesser extent the oxygen) is believed to be due to the adhesive substrate used to secure the sample.

X-ray diffraction (XRD) of the deposit showed it to be crystalline and comparison of the diffraction pattern with those of candidate reference compound patterns from the International Centre for Diffraction Data (ICDD) database showed good matches with Elemental Sulphur, S and Silicon Dioxide, SiO₂. The presence of silicon dioxide is likely to be due to the aggregate.

The SEM/EDX analysis described above for Example 18 was carried out with a deposit formed from elemental sulphur. The results are shown in Table 3:

TABLE 3

| Abundance Levels | Element |
|---|---|
| Relatively High wt % | Sulphur |
| Moderate to High wt % | Carbon |
| Low wt % | Chlorine |
| Low to trace wt % | Calcium |
| Trace wt % | Silicon, Aluminium |

Again, X-ray diffraction (XRD) of the deposit showed it to be crystalline and comparison of the diffraction pattern with those of candidate reference compound patterns from the ICDD diffraction database showed a good match with elemental sulphur, S, and with the sample from Example 18.

The EDX and XRD analysis both confirm that the precipitate from Example 18 was elemental sulphur.

### Example 26

The sulphur asphalt detection kit and method detailed above for Examples 1-25 was used to detect the presence of sulphur in sulphur concrete samples. Three sulphur concrete samples having the composition shown in Table 4 were evaluated using the sulphur detection kit and method of the present invention.

TABLE 4

| Component | Composition (wt %) |
|---|---|
| Aggregate (Normal sand) | 47% |
| Filler (Quartz) | 28% |
| Sulphur binder¹ | 25% |

¹Sulphur binder is elemental sulphur modified with bis(3-triethoxysilylpropyl) tetrasulphide modifier, as disclosed in WO2007/065920

The three samples were a conical frustrum (or a “cupcake”) crushed sulphur concrete of particle size 1-2 cm and crushed sulphur concrete or particle size <5 mm. These were treated using the sulphur detection method described above for Examples 1-25. The results are given in Table 5 below.

TABLE 5

| | Mass | Final Turbidity |
|---|---|---|
| Sample | (g) | Reading (FNU) |
| Conical frustrum | 125.6 | Over range |
| Crushed, 1-2 cm | 10.2 | 446 |
| Crushed, <5 mm | 10.2 | Over range |

With all three samples, a clearly visible white precipitate was formed and the turbidity measurements were above 400 FNU. Since the sulphur concrete samples had a higher elemental sulphur content compared to the sulphur asphalt samples it would be expected that the concentration of sulphur in the IPA extract would be higher thus leading to rapid and thick precipitate formation.

### Examples 27-30

A series of experiments were carried out varying the nature of the aqueous salt solution. Initially, the concentration of the salt solution was varied. For salt solutions such as those of calcium chloride, increasing the salt concentration correlates with increasing the ionic strength of the medium.

An asphalt sample having the same composition of that of Example 1 above was used in Examples 27-30. The sulphur detection method set out in Examples 1-25 was carried out using the sulphur detection kit detailed in Examples 1-25, except that the concentration of the calcium chloride solution was varied as set out in Table 6 below. In Example 6 water was used instead of a calcium chloride solution.

The turbidity readings (in FNU) are shown in Table 6 below.

TABLE 6

| Time | | 0.0001M | 0.001M | 0.002M |
|---|---|---|---|---|
| (Minutes) | Water | CaCl₂ | CaCl₂ | CaCl₂ |
| 5 | 144 | 176 | 437 | 481 |
| 10 | 198 | 234 | 522 | 596 |

In examples 27-30 the test solutions all turned cloudy and turbidity readings indicating the presence of sulphur were observed. Higher turbidity readings were measured when using higher concentrations of calcium chloride solution. This is due to the increased ionic strength of the aqueous solution resulting in elemental sulphur being thermodynamically unstable in the alcohol-salt solution thereby inducing precipitation.

### Examples 31-34

An asphalt sample having the same composition of sulphur detection method set out in Examples 1-25 was carried out using the sulphur detection kit detailed in Examples 1-25, except that various aqueous salt solutions were used as set out in Table 7 below.

Three different salts were examined (CaCl₂, NaCl and LiCl), each resulting in cloudy solutions being formed after addition of the alcoholic test solution. The turbidity readings (in FNU) are shown in Table 7 below.

TABLE 7

| Time | | 0.001M | 0.001M | 0.001M |
|---|---|---|---|---|
| (Minutes) | Water | CaCl₂ | NaCl | LiCl |
| 5 | 144 | 437 | 332 | 346 |
| 10 | 198 | 522 | 402 | 411 |

As can be seen from Table 7, all three salt solutions gave turbidity readings in excess of 150 FNU after 5 minutes. Calcium chloride appeared to be the most effective salt solution. From example 31 it can be seen that water was also effective in inducing precipitation of sulphur, although this occurred over a longer time span compared to the use of salt solutions.

### Examples 35-39

Examples 35-39 were carried out with a 12.3 g asphalt sample having the same composition as the asphalt of Example 25. The sulphur detection method set out in Examples 1-25 was carried out using the sulphur detection kit detailed in Examples 1-25, but with various aqueous salt solutions having varying concentrations as set out in Table 8 below. By visual inspection, each sample remained clear with no cloudiness and the turbidity readings measured were consistent with these observations.

TABLE 8

| Time | | 0.001M | 0.001M | 0.001M |
|---|---|---|---|---|
| (Minutes) | Water | CaCl₂ | NaCl | LiCl |
| 5 | 1.8 | 36.8 | 29.2 | 28.2 |

1. A method of detecting the presence of sulphur in a construction material such as asphalt or concrete comprising the steps of:
(a) adding a quantity of the construction material to a solvent to form a test solution, wherein the solvent is capable of solubilising all or part of any sulphur which is present in the construction material and wherein the solvent is miscible with an aqueous solution; and
(b) detecting the presence of sulphur in the test solution by
(i) adding to the solution a reagent which induces part or all of any sulphur present in the test solution to precipitate; and
(ii) measuring the turbidity of the test solution after the precipitate has been formed.

2. A method according to claim 1 wherein step (ii) is carried out within a time period of 2 minutes to 20 minutes after the reagent has been added to the test solution.

3. A method according to claim 1 wherein the presence of sulphur in the construction material is indicated by the test solution having a turbidity of 150 FNU or greater after the sulphur has precipitated out of solution.

4. A method according to claim 1 wherein wherein the solvent is an alcoholic solvent.

5. A method according to claim 1 wherein the reagent which induces part or all of any sulphur present in the test solution to precipitate is selected from water or an aqueous salt solution.

6. A method according to claim 5 wherein the salt of the aqueous salt solution is selected from of an alkali metal halide, an alkaline earth metal halide, an alkali metal sulphate, an alkaline earth metal sulphate, an alkali metal nitrate, an alkaline earth metal nitrate, and mixtures thereof.

7. A method according to claim 6 wherein the salt is an alkaline earth metal halide.

8. A method according to claim 7 wherein the alkaline earth metal halide is calcium chloride.

9. A method according to claim 2 wherein the turbidity of the test solution is measured using a turbidity meter.

10. A sulphur detection kit for detecting the presence of sulphur in construction materials such as asphalt or concrete wherein the sulphur detection kit comprises:
(a) solvent wherein the solvent is capable of solubilising part or all of any sulphur which is present in the construction material to form a test solution and wherein the solvent is miscible with an aqueous solution; and
(b) means for detecting the presence of sulphur in the test solution, comprising:
(i) a reagent which induces part or all of any sulphur present in the test solution to precipitate to form a turbid solution.

11. A sulphur detection kit according to claim 10 wherein the means for detecting the presence of sulphur in the test solution further comprises:
(ii) means for measuring the turbidity of the test solution after the sulphur has precipitated.

12. A sulphur detection kit according to any of claim 11 wherein the means for measuring the turbidity of the test solution after formation of the precipitate is a turbidity meter.

13. A sulphur detection kit according to claim 10 wherein the sulphur detection kit is portable.
